(19) United States
(12) Patent Application Publication
(10) Pub. No.: US 2005/0100980 A1
(43) Pub. Date: May 12, 2005

(54) Method for using saddle-point approximation for the evaluation of intractable conditional probabilities in biotechnology

(76) Inventor: Fernando J. Pineda, Baltimore, MD (US)

Correspondence Address: Francis A Cooch Office of Patent Counsel;The Johns Hopkins University, Applied Physics Laboratory, 11100 Johns Hopkins Road, Laurel, MD, 20723-6099, US

(21) Appl. No.: 10/451,020

(22) PCT Filed: Dec. 17, 2001
(86) PCT No.: PCT/US01/48801

Related U.S. Application Data
(60) Provisional application No. 60/262,623, filed on Jan. 18, 2001.

(51) Int. Cl. G06F 19/00; G01N 33/48; G01N 33/50; C12Q 1/04

(52) U.S. Cl. 435/34; 702/19

(57) ABSTRACT

A method and system for determining a probability of observing false matches between spectral peaks of an unknown source and spectral peaks of known microorganisms are provided. The method and system include using the saddle-point approximation to determine the probability of observing false matches between the spectral peaks of the unknown source and the spectral peaks of the known microorganisms. The method and system further include testing the null hypothesis to determine whether the unknown source is a known microorganism.

### CROSS-REFERENCE TO RELATED APPLICATIONS

This application claims the benefit of prior filed co-pending U.S. Application No. 60/262,623, filed Jan. 18, 2001, the disclosure of which is hereby incorporated herein by reference.

### BACKGROUND OF THE INVENTION

1. Field of the Invention

The present invention relates to microorganism identification. More specifically, the present invention relates to a method for quantifying false matches between spectral peaks of an unknown source and spectral peaks of known microorganisms using saddle-point approximation.

2. Description of the Related Art

Proteins expressed in microorganisms can be used as biomarkers for microorganism identification. In particular, mass spectra obtained by matrix-assisted laser desorbtion/ionization (MALDI) time-of-flight (TOF) instruments have been employed for rapid microorganism differentiation and classification. The identification is based on differences in the observed “fingerprint” protein profiles for different organisms, typically in the mass range 4-20 kDa. A crucial requirement for successful identification via fingerprint techniques is spectral reproducibility. However, mass spectra of complex protein mixtures depend in an intricate and oftentimes poorly characterized fashion on a number of factors including sample preparation and ionization technique (e.g., MALDI matrixes, laser fluence), bacterial culture growth times and media, etc.

It has been proposed to exploit the wealth of information contained in prokaryotic genome and proteome databases to create a potentially more robust approach for mass spectrometry-based microorganisms identification (See Demirev, P. A.; Ho, Y. P.; Ryzhov, V.; Fenselau, C., Anal. Chem 1999, 71, 2732-8). This approach is independent of the chosen ionization and mass analysis model. The central idea of this proposed approach is to match the peaks, in the spectrum of an unknown microorganism, with the annotated proteins of known microorganisms in a proteomic database (e.g., the internet-accessible SWISS-PROT proteomic database).

The plausibility of the proposed approach was demonstrated by identifying two microorganisms whose genomes are known (B. subtilis and E. coli). The identification was performed by assigning a matching score, k, to each microorganism. This score was simply the number of spectral peaks that matched (to within a specified mass tolerance) the annotated proteins of each of the microorganisms in the database. The microorganisms were subsequently ranked according to their score, and the microorganism with the highest score was declared to be the unknown source of the spectrum.

Although this simple ranking algorithm succeeded in correctly identifying two microorganisms from a relatively small database, it was nonetheless understood from the onset that more rigorous methods would be necessary to perform robust identification of a broader range of microorganisms over more comprehensive databases. A key component of robust microorganism identification must be the ability to quantitatively assess the risk of false identification. In the present setting, false identification can occur when a large number of spectral peaks accidentally match the masses of proteins in the proteome of an unrelated microorganism. The likelihood of accidental matches, and hence the likelihood of false identification, increases, if the mass tolerance is increased or if the size of the known proteome increases.

In general, it is impractical to estimate the risk of false identification by exhaustively performing a large number of proteome-spectrum comparisons with a large number of experimentally obtained spectra. Instead, it is necessary to base quantitative methods on models of the matching and measurement processes.

Accordingly, a need exists to develop, validate and apply an algorithmic model of the matching and measurement processes and use it to estimate the likelihood of misidentification and to gain insight into the nature of the microorganism identification problem.

A previous patent application having U.S. application Ser. No. 06/196,368 and filed on Apr. 12, 2000 with the title “Method and System for Microorganism Identification by Mass Spectrometry-based Proteome Database Searching” describes a method of quantifying the significance of microorganism identification by introducing a false match model and a scoring algorithm based on p-values. The key to the false match model was the simplifying assumption that the proteins in a microorganism's proteome were uniformly distributed in the mass range of interest. This allowed one to calculate the expected number of matches between the peaks in a mass spectrum and the peaks in a proteome. Thus, one could easily test the null hypothesis that the mass spectrum was not generated by the microorganism in question.

### SUMMARY OF THE INVENTION

The present invention extends the previously disclosed method of quantifying the significance of microorganism identification by permitting non-uniform distributions of masses. The p-value calculations can be computationally intensive. Thus, saddle-point approximation is introduced to numerically evaluate the p-values. The saddle point approximation allows the efficient testing of the null hypothesis that the mass spectrum was not generated by the microorganisms in question.

### DESCRIPTION OF THE PREFERRED EMBODIMENTS

To assess the likelihood of false identification, the present invention derives a model-based distribution of scores due to false matches. For a given known microorganism with a corresponding annotated proteome, the inventive model denotes this distribution as P_{K}(k), where K is the number of peaks in the spectrum of the unknown and k is the number of these peaks that match proteins in the proteome.

The distribution P_{K}(k) allows testing of the significance of the scores via hypothesis testing and allows for quantifying the scalability of the approach by establishing limits on the size of the database (number of individual proteomes) and on the size of the proteomes in the database. Finally, the null hypothesis, Hₒ, is tested that the unknown and the known microorganisms are not the same.

An approximate probability distribution will now be derived for observing exactly k false matches when a spectrum from an unknown microorganism is compared to the proteome of a known microorganism according to the invention. In the mass range [mₘᵢₙ, mₘₐₓ], the spectrum is assumed to have K peaks and the proteome is assumed to have n proteins.

The database contains a label and a corresponding mass list for each potentially observable microorganism. It is understood that the proteomes in the database are neither necessarily complete, nor error free. Nevertheless, the inventive method assumes that each mass list is sufficiently inclusive and sufficiently accurate, that it is reasonable to expect that some of the masses in the mass list will be found in a physical mass spectrum. In such a setting it is reasonable to compare a spectrum to a mass list.

The spectrum from an unknown source is compared to the mass list of a known object by matching spectral peaks against masses in the mass list. A database hit occurs when the mass of a protein in the database differs from the mass of a spectral peak by at most Δm/2. A spectral peak with one or more database hits is said to be a “matched peak”. The number of spectral peaks that match masses in a mass list is said to be the “score” of the object.

To derive the approximate distribution of false matches, assume that the unknown source (s) and the known object (t) are distinct (i.e., s≠t). Then, by definition, all matches are false matches. We make no assumptions about the distributions of masses throughout the mass range [mₘᵢₙ, mₘₐₓ]. It is straightforward to write down P_{match}, which is the probability that a given peak will be a matched peak. In particular, given any interval of width Δm about a mass m, the probability P(q) of obtaining exactly q database hits is Poisson distributed:

$$P ( q ) = \frac{{( ρ ( m ) Δ m )}^{q} {ⅇ}^{- p ( m ) Δ m}}{q !} , \qquad ( 1 )$$

where ρ(m) is the density of proteins in the proteome in the mass range [mₘᵢₙ, mₘₐₓ]. Consequently, the probability of obtaining no database hits is P(0)=exp(−ρΔm) and the probability of obtaining at least one database hit for the I-th mass in the list is
pᵢ≡1−P(0)≡1−e^{-ρ(m}^{ᵢ}^{)Δm}. (2)
Let cᵢ be a binary random variable that is 1 if the i-th peak has a match and zero otherwise. Then, the probability of a particular configuration of matches {c₁, . . . , c_{K}} is a multivariate Bernoulli distribution

$${P}_{K} ( c ) = {\prod }_{i = 1}^{K} {{p}_{i}^{{c}_{i}} ( 1 - {p}_{i} )}^{1 - {c}_{i}} . \qquad ( 3 )$$

From this the probability of exactly k false matches is

$$P ( k ) = {\sum }_{\vert c \vert = k}^{} {P}_{K} ( c ) \qquad ( 4 )$$

where the sum is over all terms that have

$${\sum }_{i}^{} {c}_{i} = k . The corresponding p \text{-} value is$$

$$α = {\sum }_{k > {k}_{observed}}^{} {P}_{K} ( k ) . \qquad ( 5 )$$

In general P_{K}(k) is computationally intractable. But P_{K}(k) is tractable if (1) the number of peaks, K, is small; (2 pᵢ=p for all i (uniform approximation); and (3) the number of peaks, K, is large (saddle-point approximation).

The saddle point approximation for P_{K}(k) is

$${P}_{K} ( k ) \approx \{ {\prod }_{i = 1}^{K} {p}_{i} ( 1 - {p}_{i} ) \} \cdot \frac{exp ( Kf ( μ ) )}{\sqrt{2 π {\sum }_{j = 1}^{K} {σ}^{′} ( {h}_{j} + μ )}} \qquad ( 6 )$$

where μ is the unique solution of

$$\begin{matrix}\begin{matrix}f ( μ ) ≡ - ( \frac{k}{K} ) μ + \frac{1}{K} {\sum }_{j = 1}^{K} log ( 1 + exp ( {h}_{j} + μ ) ) \\ where\end{matrix} & ( 7 ) \\ \begin{matrix}k = {\sum }_{j = 1}^{K} σ ( {h}_{j} + μ ) \\ and where\end{matrix} & ( 8 ) \\ {h}_{i} ≡ log ( \frac{{p}_{i}}{1 - {p}_{i}} ) & ( 9 )\end{matrix}$$

To conclude, the present invention quantifies the significance of microorganism identification by mass spectrometry-based proteome database searching through the use of a statistical model of false matches and saddle-point approximation.

What has been described herein is merely illustrative of the application of the principles of the present invention. For example, the functions described above and implemented as the best mode for operating the present invention are for illustration purposes only. Other arrangements and methods may be implemented by those skilled in the art without departing from the scope and spirit of this invention.

1. A method for determining a probability of observing false matches between spectral peaks of an unknown source and spectral peaks of known microorganisms, said method comprising the steps of:
providing a proteomic database for storing data of known microorganisms;
determining the spectral peaks of known microorganisms using the proteomic database;
comparing the spectral peaks of the unknown source with the spectral peaks of the known microorganisms; and
using the saddle-point approximation to determine the probability of observing false matches between the spectral peaks of the unknown source and the spectral peaks of the known microorganisms.

2. The method according to claim 1, further comprising the step of testing the null hypothesis that the unknown source is a known microorganism.

3. A method for determining a probability of observing false matches between spectral peaks of an unknown source and spectral peaks of known microorganisms, said method comprising the step of:
using the saddle-point approximation to determine the probability of observing false matches between the spectral peaks of the unknown source and the spectral peaks of the known microorganisms.

4. The method according to claim 3, further comprising the step of testing the null hypothesis that the unknown source is a known microorganism.

5. A system for determining a probability of observing false matches between spectral peaks of an unknown source and spectral peaks of known microorganisms, said system comprising:
means for providing a proteomic database for storing data of known microorganisms;
means for determining the spectral peaks of known microorganisms using the proteomic database;
means for comparing the spectral peaks of the unknown source with the spectral peaks of the known microorganisms; and
means for using the saddle-point approximation to determine the probability of observing false matches between the spectral peaks of the unknown source and the spectral peaks of the known microorganisms.

6. The system according to claim 5, further comprising means for testing the null hypothesis that the unknown source is a known microorganism.
